(19) United States
(12) Patent Application Publication
(10) Pub. No.: US 2022/0000576 A1
(43) Pub. Date: Jan. 6, 2022

(54) CAPSULE DEVICE TO ENCASE A BODY ORGAN OR MASS AND USE THEREOF

(71) Applicants: Capsulacare LLC, Pittsburgh, PA (US); Fujita Academy, Toyoake-Shi (JP)

(72) Inventors: Kyongtae BAE, Pittsburgh, PA (US); Junu BAE, Pittsburgh, PA (US); Sonu BAE, Pittsburgh, PA (US); Shizuko NAGAO, Toyoake-Shi (JP); Kanako KUMAMOTO, Toyoake-Shi (JP); Aya YOSHIMURA, Toyoake-Shi (JP); Tamio YAMAGUCHI, Suzuka-shi (JP)

(21) Appl. No.: 17/289,440

(22) PCT Filed: Oct. 29, 2019
(86) PCT No.: PCT/US2019/058605

§ 371 Date: Apr. 28, 2021

Related U.S. Application Data
(60) Provisional application No. 62/756,358, filed on Nov. 6, 2018.

(51) Int. Cl. A61B 90/00

(52) U.S. Cl. CPC A61B 90/08; A61B 2090/0815

(57) ABSTRACT

A method for treatment or prevention of diseases accompanying abnormal growth of a body organ or mass. Specifically, a method of manufacturing a capsule device to encase a body organ such as kidney, liver and ovary is provided. Abnormal growth of a body organ is slowed or halted by covering the body organ such as kidney, liver and ovary. The diseases associated with abnormal growth include, for example, polycystic kidney disease and polycystic liver disease.

### RELATED APPLICATION

This application claims priority to U.S. Provisional Application Ser. No. 62/756,358, entitled “CAPSULE DEVICE TO ENCASE A BODY ORGAN OR MASS AND USE THEREOF”, filed Nov. 6, 2018, which is incorporated herein by reference in its entirety.

### TECHNICAL FIELD

The present invention relates to a capsule device to encase a body organ, such as kidney, liver or ovary, or a mass, as well as a method of manufacturing the capsule device. More particularly, it relates to a capsule device used for slowing or stopping abnormal growth of body organs or masses and a method of manufacturing the same. The invention also relates to the use of a capsule device in the treatment or prevention of diseases associated with abnormal growth of body organs or masses, and to a method for the treatment or prevention of diseases associated with abnormal growth of body organs or masses.

### BACKGROUND

Autosomal Dominant Polycystic Kidney Disease (ADPKD)

Autosomal dominant polycystic nephropathy (ADPKD) is the most common hereditary renal disorder, with episodes occurring in any ethnic group. The prevalence rate is one in 400 to 1,000 live births (i.e., 12.5 million people worldwide), and it is the fourth leading cause worldwide of chronic kidney disease (CKD) (Non-Patent Literatures 1 to 3). ADPKD initiates in utero due to mutations in PKD1 (encoding polycystin 1) and PKD2 (encoding polycystin 2). PKD1 accounts for 85% of cases, while PKD2 accounts for the remaining cases. The disease is phenotypically characterized by bilateral enlarged kidneys containing numerous cysts that expand and compress surrounding renal parenchyma. On average, half of the patients with ADPKD progress to end-stage renal disease (ESRD) by 60 years of age. However, there are significantly high phenotypic variabilities, ranging from newborns with massive cystic kidneys to patients whose kidney function remain relatively normal well into old age. Mutations in PKD1 leads to more severe disease (average age of ESRD onset of 58.1 years) than PKD2 (that of 79.7 years). Aside from the genetic factors, many clinical (sex, history of early hypertension and urological adverse events, number of pregnancies) and environmental modifiers affect the disease severity.

In addition to ESRD, patients with ADPKD suffer from a number of life-altering symptoms associated with the progression of disease, including hypertension, abdominal pain and expansion from cysts, hematuria, and impaired quality of life. Reduction of these symptoms and delay of progressive loss of kidney function or the onset of ESRD are significant in improving the quality of life in patients with ADPKD. Thus, until a complete cure is discovered, the goal of disease management for these patients is focused on the development of personalized care strategies to create meaningful changes in clinical outcomes and delay the progression of disease.

Imaging of ADPKD and Kidney Volume

Radiological imaging is essential in the diagnosis of ADPKD, particularly in situations of positive family history of the disease. In some cases, the diagnosis can be made by genetic testing. Patients with ADPKD are typically described in imaging with bilateral large kidneys containing multiple bilateral cysts that progressively increase in size and number. The imaging diagnostic criteria is established by considering factors including imaging modality, family history of ADPKD, age of patient, and number of kidney cysts. Disease severity is reflected by the number and size of cysts that cause the overall volume of the kidney to expand while depleting the amount of normal renal parenchyma (Non-patent Literature 4). Secondary complications of ADPKD (for example, pain, hypertension and gross hematuria) begin in childhood and eventually affect most patients by the fifth decade of life. These complications increase in frequency in patients with larger kidneys.

Quantitative radiological imaging is an invaluable tool to measure ADPKD kidney and cyst growth as markers for monitoring the rate of disease progression and response to therapy (Non-patent Literature 4). In particular, measuring the size of cysts and kidneys provides an important index by which to gauge the efficacy of measures targeted at reducing abnormal growth. Radiological imaging with ultrasonography, CT and MRI can be used to quantify the rate of kidney volume growth. While CT and MRI are the most accurate techniques for volumetric imaging, ultrasonography is also useful for initial screening and size estimation. Kidney volume information obtained from radiological images can be used to guide clinical management of patients with ADPKD such as ADPKD-related risk assessment. Accurate risk stratification is required for the prescription of treatments to determine whether the benefits of treatment outweigh the therapeutic burden of costs and adverse effects.

Pharmaceutical Treatments for ADPKD

At present, there is no cure for ADPKD. Treatment to prevent or slow down the progression of ADPKD has not been well established. Various dietary regimens including low-salt, low-protein diet and high-water intake were investigated but determined to have limited efficacy. Multiple targeted pharmaceutical therapies were explored, from reno-protective drugs, such as angiotensin-converting-enzyme inhibitors and angiotensin-receptor blockers, to agents that specifically target molecular pathways involved in the formation and expansion of cysts, such as vasopressin-receptor blockers, somatostatin, and targets of the rapamycine (mTOR) pathway. These pharmaceutical therapeutic agents for ADPKD showed variable degrees of promising results but have not been widely accepted in part because of the range of adverse drug effects and the requirement for the patient to continually administer the drug in order to be effective.

An additional difficulty in evaluating the efficacy of pharmaceutical treatment is that the disease often begins in utero and progresses very slowly throughout life. The onset of ADPKD symptoms and the insurgence of renal dysfunction may extend over decades. Renal functional biomarkers such as urinary albumin and glomerular filtration rate (GFR) have no predictive value in the early stages of the disease and only become useful indicators of renal function after major, irreversible damage has been done to renal function. Even with promising candidates for targeted ADPKD therapy, there is no marker of disease progression that enables potential treatments to be tested in early stages of the disease. The lack of such markers forces therapeutic trials of patients with ADPKD to focus on late stages of the disease process, in which there are unequivocal signs of reduced GFR. However, at this stage, other secondary processes, such as inflammation and fibrosis, which are seen in most end-stage renal disorders, arise to obscure the impact of therapies directed at specific ADPKD pathogenetic targets. The desire to test novel potential therapies has urged the discovery of quantifiable markers of ADPKD progression such as kidney volume measurement that can be safely used in children and in adults.

Surgical Treatments for ADPKD

Surgical treatments for ADPKD are focused mainly on managing the short-term clinical complications of the disease and not on preventing the progression of the disease or renal failure (Non-patent Literature 5). When renal cysts are infected or significantly enlarged to cause abdominal pain, image-guided percutaneous aspiration of cysts with or without sclerotherapy is the first-line treatment. Recurrent or aspiration-resistant cysts may require open- or laparoscopic-guided surgical decortication to excise the outer walls and to drain them. Surgical decortication of cysts is shown to be highly effective in the management of disease-related chronic pain for the majority of patients with ADPKD (Non-patent Literature 6). However, a potential role of surgical cyst decortication to alleviate hypertension or preserve renal function has not been established. Furthermore, ADPKD patients who have symptomatic inaccessible cysts in the medullary portions of the kidney may not gain pain relief from surgical decortication of cortical cysts. In these patients, nephrectomy is the last resort to control the pain. Nephrectomy is also performed to create space for renal allograft in some APDKD patients with massively enlarged kidneys who undergo renal transplantation. In addition, transcatheter renal artery embolization is used to reduce renal volume and improve lung function in ADPKD patients with nephromegaly who are on hemodialysis (Non-patent Literature 7).

Other Non-Renal Visceral Cystic Diseases and Mass

In addition to polycystic kidney disease, abnormal formation and growth of cysts can affect the integrity and function of other organs including liver and ovary. In particular, polycystic liver disease (PCLD) can occur commonly in patients with ADPKD. PCLD is characterized by multiple diffuse cystic lesions of the liver parenchyma. The volumes of the liver and liver cysts are important disease biomarkers for the assessment of the severity of polycystic liver disease. The cysts may vary considerably in number and size and may cause marked enlargement of the liver. Patients with massive liver enlargement may develop symptoms related to compression of surrounding organs including abdominal and back pain, abdominal distension, dyspnea, early satiety, and early post-prandial fullness, inferior vena cava or hepatic venous-outflow obstruction. The main treatment of symptomatic PCLD patients is surgical therapy including laparoscopic- or open-fenestration that is aimed to significantly reduce the size of the polycystic liver and provide long-term relief of symptoms without compromising liver function (Non-patent Literature 8). Some patients may undergo partial resection of liver or transplantation of liver to relieve debilitating symptoms. A novel method to reduce the volumetric growth of the polycystic liver may provide an alternative approach to the current surgical therapy to patients with symptomatic PCLD.

Polycystic ovarian disease is also characterized by enlarged bilateral ovaries with the presence of multiple peripheral cysts. Clinical management includes lifestyle modifications, pharmacotherapy, and surgical intervention. Surgical management of polycystic ovarian disease is aimed mainly at restoring ovulation. Various laparoscopic methods such as electrocautery and laser drilling are used to treat the ovarian cortex and stroma. Surgical complications include formation of adhesions and ovarian atrophy. In addition, a lump or a bump called mass sometimes occurs in a part of a body or an organ of a mammal including human beings. The term mass may include tumors, inflammatory lesions, bulging blood vessels, and other unspecified lumps.

### RELATED ART DOCUMENTS

### Non-Patent Literatures

[Non-patent Literature 1] Grantham, J. J., Clinical practice. Autosomal dominant polycystic kidney disease. N Engl J Med, 2008. 359(14): p. 1477-85.
[Non-patent Literature 2] Grantham, J. J., S. Mulamalla, and K. I. Swenson-Fields, Why kidneys fail in autosomal dominant polycystic kidney disease. Nat Rev Nephrol, 2011. 7(10): p. 556-66.
[Non-patent Literature 3] Chebib, F. T. and V. E. Torres, Autosomal Dominant Polycystic Kidney Disease: Core Curriculum 2016. Am J Kidney Dis, 2016. 67(5): p. 792-810.
[Non-patent Literature 4] Bae, K. T. and J. J. Grantham, Imaging for the prognosis of autosomal dominant polycystic kidney disease. Nat Rev Nephrol, 2010. 6(2): p. 96-106.
[Non-patent Literature 5] Akoh, J. A., Current management of autosomal dominant polycystic kidney disease. World J Nephrol, 2015. 4(4): p. 468-79.
[Non-patent Literature 6] Millar, M., et al., Surgical cyst decortication in autosomal dominant polycystic kidney disease. J Endourol, 2013. 27(5): p. 528-34.
[Non-patent Literature 7] Yamakoshi, S., et al., Transcatheter renal artery embolization improves lung function in patients with autosomal dominant polycystic kidney disease on hemodialysis. Clin Exp Nephrol, 2012. 16(5): p. 773-8.
[Non-patent Literature 8] Russell, R. T. and C. W. Pinson, Surgical management of polycystic liver disease. World J Gastroenterol, 2007. 13(38): p. 5052-9.

### SUMMARY OF THE INVENTION

### Problem to be Solved by the Invention

One of the objects of the present invention is to provide a capsule device to encase a body organ, such as kidney, liver or ovary, or a mass, as well as a method of manufacturing the capsule device. Another object of the invention is to provide the use of a capsule device in the treatment or prevention of diseases associated with abnormal growth of body organs or masses, as well as a method for the treatment or prevention of diseases associated with abnormal growth of body organs or masses.

### Solution to the Problem

The present inventors developed a capsule device to encase a body organ, such as kidney, liver or ovary, or a mass. Using this capsule device allows the treatment or prevention of diseases accompanying abnormal growth of body organs such as polycystic kidney disease (e.g., ADPKD) and the like. The invention encompasses the following embodiments:

[Embodiment 1] A capsule device comprising: a body having an inner cavity for encasing a body organ or mass.

[Embodiment 2] The capsule device according to Embodiment 1, wherein the capsule device has a shape approximating to the body organ or mass.

[Embodiment 3] The capsule device according to Embodiment 1 or 2, wherein the body organ is selected from the group consisting of kidney, liver, and ovary.

[Embodiment 4] The capsule device according to any one of Embodiments 1 to 3, wherein the capsule device is used for slowing down or halting growth of the body organ or mass.

[Embodiment 5] The capsule device according to any one of Embodiments 1 to 4, wherein the capsule device is configured to have an aperture to ensure that a structure connected to the body organ or mass is not interfered.

[Embodiment 6] The capsule device according to any one of Embodiments 1 to 5, wherein the body organ is kidney.

[Embodiment 7] The capsule device according to Embodiment 6, wherein the capsule device is used for the treatment or prevention of polycystic kidney disease.

[Embodiment 8] The capsule device according to Embodiment 6 or 7, wherein the capsule device is designed to cover substantially the entire kidney.

[Embodiment 9] The capsule device according to any one of Embodiments 6 to 8, wherein the capsule device is designed to suppress the increase of total kidney volume.

[Embodiment 10] The capsule device according to any one of Embodiments 6 to 9, wherein the capsule device is designed not to interfere with the renal artery, renal vein, and ureter.

[Embodiment 11] The capsule device according to any one of Embodiments 1 to 10, wherein the capsule device is produced by personalized 3-D fabrication of the capsule device on the basis of medical imaging data of a subject.

[Embodiment 12] The capsule device according to Embodiment 11, wherein the personalized 3-D fabrication is performed using automated 3-D printing or manual fabrication.

[Embodiment 13] The capsule device according to Embodiment 11 or 12, wherein the medical imaging data is obtained using MRI, CT, ultrasound images, fluoroscopic images, or laparoscopic images.

[Embodiment 14] The capsule device according to any one of Embodiments 1 to 13, wherein bio-compatible material, elastic property, configuration, and/or size of the capsule device are determined based on an individual subject's medical information selected from the group consisting of: subject's age, subject's gender, subject's allergic sensitivity profile, target organ's anatomy, and target organ's projected growth rate.

[Embodiment 15] The capsule device according to any one of Embodiments 1 to 14, wherein the capsule device is configured to include predetermined surgical opening and closing suture lines within the device by consideration of surgical procedures for the implantation of the capsule device.

[Embodiment 16] The capsule device according to any one of Embodiments 1 to 15, wherein the capsule device is designed to split open at least partially during the placement in order to encase the organ.

[Embodiment 17] The capsule device according to Embodiment 16, wherein the capsule device comprises a means to close the split openings of the device.

[Embodiment 18] The capsule device according to Embodiment 17, wherein the closing means is selected from the group consisting of interlacing strings, buttons, hooks, fasteners, and hook-and-loop fasteners.

[Embodiment 19] The capsule device according to any of the Embodiments 1 to 14, wherein the capsule device is made of liquid or flexible injectable material capable of being implanted by minimally-invasive or laparoscopic surgical procedures.

[Embodiment 20] The capsule device according to Embodiment 19, wherein the injectable material is capable of being implanted by minimally-invasive or laparoscopic surgical procedures.

[Embodiment 21] A method of producing the capsule device according to any one of Embodiments 1 to 18, comprising:

measuring the shape of a body organ or mass of a subject;
designing a capsule device adapted for the body organ or mass;
fabricating the capsule device.

[Embodiment 22] The method according to Embodiment 21, wherein the measuring is performed using MRI, CT, ultrasound images, fluoroscopic images, or laparoscopic images.

[Embodiment 23] The method according to Embodiment 21 or 22, wherein the fabricating is performed using 3-D printing or manual fabrication.

[Embodiment 24] The method according to any one of Embodiments 21 to 23, wherein the designing comprises determining bio-compatible material, elastic property, configuration, and/or size of the capsule device based on individual subject's medical information selected from the group consisting of: subject's age, subject's gender, subject's allergic sensitivity profile, target organ's anatomy, and target organ's projected growth rate.

[Embodiment 25] The method according to any one of Embodiments 21 to 24, wherein the designing comprises including predetermined surgical opening and closing suture lines within the device by consideration of efficient surgical procedures for the implantation of the device.

[Embodiment 26] The method according to any one of Embodiments 21 to 25, wherein the designing comprises designing the capsule device to be split open at least partially in order to encase the organ or mass.

[Embodiment 27] The method according to Embodiment 26, wherein the designing comprises including a means to close the split openings of the device.

[Embodiment 28] The method according to Embodiment 27, wherein the means to close the split openings of the device is selected from the group consisting of interlacing strings, buttons, hooks, fasteners, and hook-and-loop fasteners.

[Embodiment 29] The method according to Embodiments 21 to 24, wherein the designing comprises selecting liquid or flexible injectable material for implanting and fabricating the capsule devices via minimally-invasive or laparoscopic surgical procedures.

[Embodiment 30] A method for the treatment or prevention of abnormal growth of a body organ or mass in a subject in need thereof, comprising:

implanting the capsule device of Embodiments 1 to 20 to encapsulate the body organ or mass of the subject.

[Embodiment 31]

The method of Embodiment 30, wherein the body organ is selected from the group consisting of kidney, liver, and ovary.

[Embodiment 32] The method according to Embodiment 30 or 31, wherein the body organ is kidney.

[Embodiment 33] The method according to Embodiment 32, wherein the abnormal growth is caused by ADPKD or ARPKD.

[Embodiment 34] A use of the capsule device of Embodiments 1 to 20 in the treatment or prevention of abnormal growth of a body organ or mass.

[Embodiment 35] The use according to Embodiment 34, wherein the body organ is selected from the group consisting of kidney, liver, and ovary.

[Embodiment 36] The use according to Embodiment 35, wherein the body organ is kidney.

[Embodiment 37] The use according to Embodiment 36, wherein the abnormal growth is caused by ADPKD or ARPKD.

### BRIEF DESCRIPTION OF THE DRAWINGS

FIG. 1A shows an overall image of a capsule device 1 for a kidney. This capsule device is hollow, has a size suitable for enclosing the kidney, and has an aperture 2 to ensure that tubular structures connected to the kidney are not interfered. Further, the capsule device may be provided with a suture line 3 used when encasing the kidney.

FIG. 1B shows an overall image of the capsule device 1 having broadened opposite facing edges 4. In this exemplary figure, the device has been split-opened along the suture line 3 and the overlapping edges 4 are utilized to reinforce the closure of split openings, for example by suturing, threading without needle puncture, or gluing. This figure shows that the edges 4 are provided with holes 5 for facilitating suturing, and that the split sections can be closed by suture thread 6.

FIG. 1C shows an overall image of the capsule device 1 having broadened opposite facing edges 4 in a split-opened configuration. In this exemplary figure, the device has been split-opened along the suture line 3 and the overlapping edges 4 are utilized to reinforce the closure of split openings, for example by suturing or gluing. This figure also shows that holes 5 may be provided in the broadened edges 4 to facilitate suturing or threading without needle puncture.

FIG. 1D shows a capsule device made of silicone rubber with opposite facing broadened edges in a split-open configuration.

FIG. 2 is a flowchart showing an overall scheme exemplifying a treatment method of ADPKD.

FIG. 3 shows folding of a capsule device. First, a folded capsule device is stored in a catheter that is inserted into a body cavity to deliver the capsule device to the target kidney. The folded capsule device is subsequently released from the catheter and expanded in the peritoneal cavity before being placed on the target kidney.

FIG. 4 shows the encasing of a kidney by a capsule device. An elastically stretchable capsule device can extend and widen its opening. After the lower pole of the kidney is placed, the device is mobilized and stretched further to encase the entire volume of the kidney.

FIG. 5 shows an example of a suture line on a capsule device. In this example, the capsule device is notched vertically and horizontally in advance in order to facilitate the encasement of the entire kidney, and a string that connects each part of the device is attached in a manner like a shoelace. After the placement of the capsule device over the kidney, the connecting strings are pulled back to juxtapose the sections and close over the entire kidney volume.

FIG. 6 shows a capsule device fabricated by 3D printing. Two different sized (medium and large) rat kidney capsule devices are shown.

FIG. 7 shows a capsule device placed over the left kidney of a PKD rat and bilateral kidneys from the same rat.

FIG. 8 shows bilateral kidneys from 3 PKD rats. Top: Kidneys derived from a PKD rat with capsule devices placed over bilateral kidneys. Middle: Kidneys from a PKD rat with a capsule device only in the left kidney. Bottom: Kidneys from a PKD rat subjected to bilateral sham operations.

FIG. 9 shows the right kidney of a wild-type rat before (left) and after (right) the placement of a capsule device.

FIG. 10 shows the right kidney (left) of Cy/+ rats without the capsule device and the left kidney (right) of the same rat with the capsule device attached. The weights of the right kidney and the left kidney were 6.14 g and 3.91 g, respectively.

FIG. 11 shows longitudinal cross-sections (H&E staining) of the right kidney (left) of a Cy/+ rat not encased in a capsule device and the left kidney (right) of the same rat encased in a capsule device.

FIG. 12 shows images of a magnified cortex segment of longitudinal cross-sections (H&E staining) of the right kidney (left) of a Cy/+ rat not encased in a capsule device and the left kidney (right) of the same rat encased in a capsule device. The left kidney with the capsule device shows that the intrinsic anatomical renal capsule is thickened.

FIG. 13 shows images of histological sections (H&E staining) of the right kidney (left) of a Cy/+ rat not encased in a capsule device and the left kidney (right) of the same rat encased in a capsule device. The capsuled left kidney shows that the size of cysts is suppressed.

FIG. 14 shows images of sections stained for Ki67 of the right kidney (left) of a Cy/+ rat not encased in a capsule device and the left kidney (right) of the same rat encased in a capsule device. The capsuled left kidney shows that the presence of Ki67 (indicated by arrows) is reduced.

### DETAILED DESCRIPTION OF THE INVENTION

As mentioned above, the present inventors developed a capsule device to be implanted into a living body to encase a body organ or mass, which can be used to slow down or stop the growth, including abnormal growth of the body organ or mass. The organs to which the capsule device of the present invention may be applied include kidney, liver, and ovary. In addition, the mass refers to a lump or a bump that occurs in a part of a body or an organ, and may include tumors, inflammatory lesions, bulging blood vessels, and other unspecified lumps. The present invention is described in more detail below.

Method for Treatment of ADPKD and Device Used Therefor

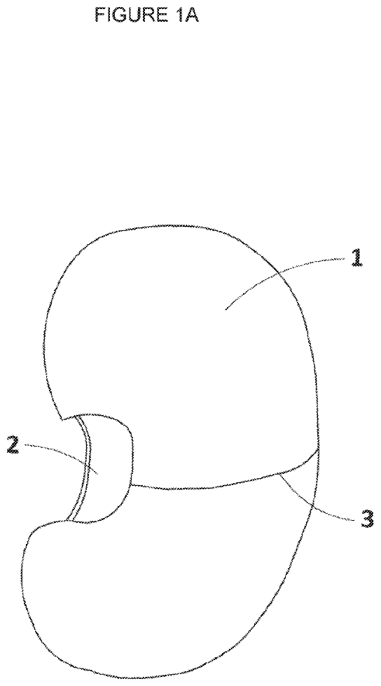

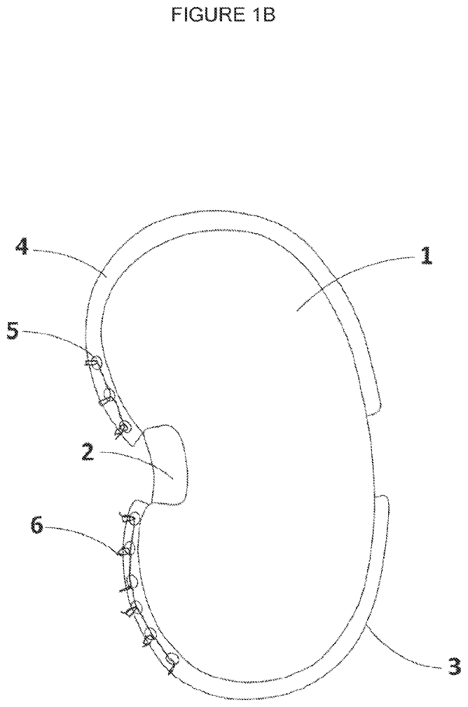

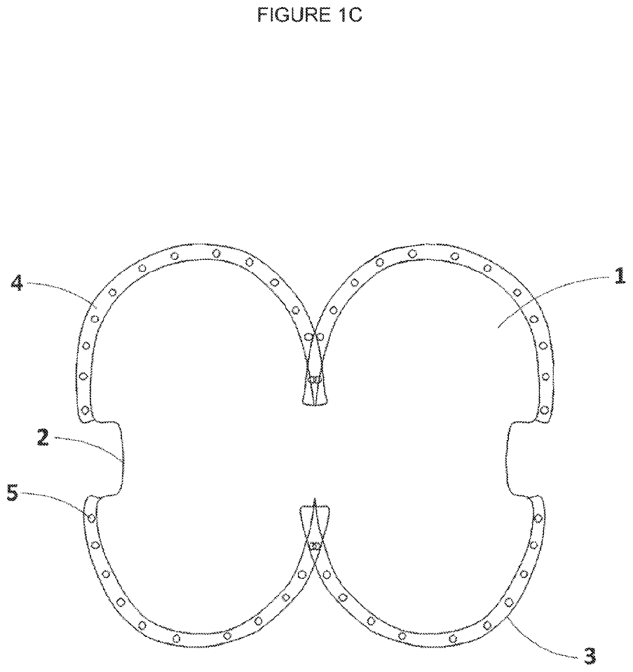

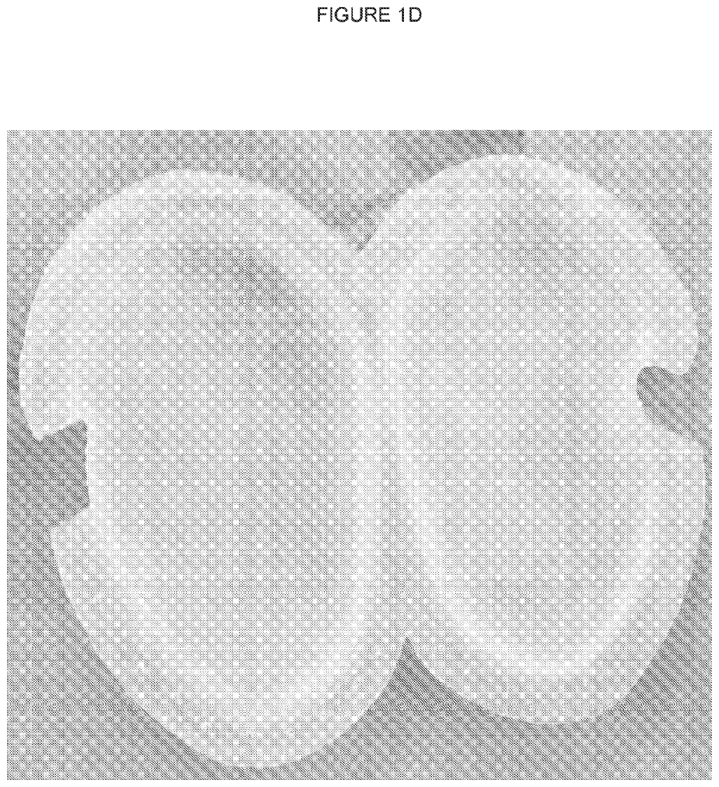

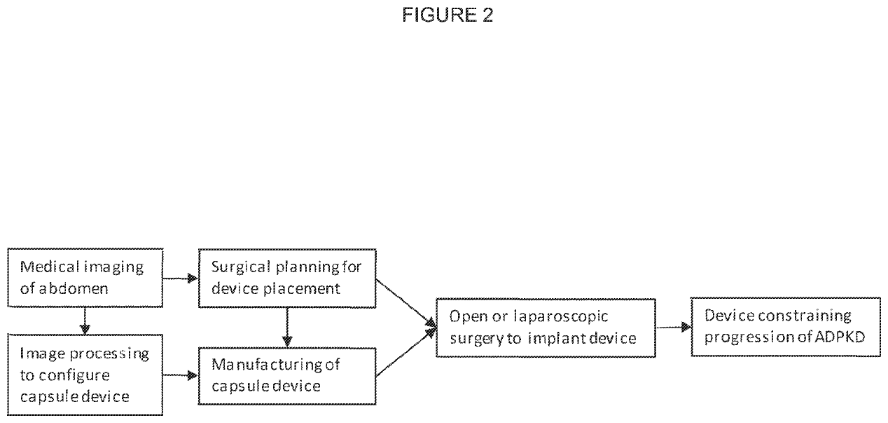

An exemplary capsule device for application to the kidney is shown in FIG. 1. This figure shows an overall image of a capsule device 1 for a kidney. This capsule device is hollow, has a size suitable for enclosing the kidney, and has an aperture 2 to ensure that tubular structures connected to the kidney are not interfered. Further, the capsule device may be provided with a suture line 3 used when encasing the kidney. Thus, the capsule device of the present invention may have a body having an inner cavity for encasing a body organ or mass. An overall scheme of a method for treating or preventing abnormal growth of a body organ according to the present invention is shown in FIG. 2, taking as an example a treatment method of ADPKD.

Rationale for Constraining Volumetric Expansion of Kidney to Treat ADPKD

As mentioned above, several diseases associated with abnormal growth of animal bodily organs are known. For example, cystic kidney disease (cystic kidney) is known as a disease that causes an abnormal increase in kidney volume. Polycystic kidney disease (PKD) is an inherited disorder mainly classified into autosomal dominant polycystic kidney disease (ADPKD) and autosomal recessive polycystic kidney disease (ARPKD); cysts are developed in kidney (100%) and liver (male 60 to 70%, female 80%), as well as in pancreas, spleen, uterus, testicles, seminal vesicles, and abnormal cell proliferation, inflammation, fibrosis and cyst fluid accumulation are observed. Autosomal dominant polycystic kidney disease (ADPKD) is a disorder characterized by numerous cysts derived from the renal tubule being randomly expanded, and this cystic growth often results in abnormal kidney hypertrophy. These cysts cause secondary complications including pain, hypertension and gross hematuria. Renal failure is usually not detected until the patient's age reaches 50 to 60 years. In animal models of this disease, it has been shown that treatment targeting molecules and pathophysiological abnormalities can delay cyst growth and protect kidney function. Unfortunately, the transition to clinical trials of these treatments is not progressing. The reason is that the glomerular filtration rate, a common biomarker of kidney disease progression, does not substantially decrease until extensive irreversible damage to the noncystic parenchyma occurs. On the other hand, ultrasonography, CT and MRI have been used for many years to quantify kidney volume increase in patients with ADPKD. Imaging using these techniques is also used to accurately quantify the rate of increase in the patient's kidney and total cyst volume.

Kidney volume is closely associated with the severity of clinical symptoms and renal function in patients with ADPKD. From the perspective of structural disease progression, reducing kidney volume would be beneficial to relieve some disease-related symptoms in ADPKD patients, even if it only prevented the grotesque enlargement of organs. Experimental studies in animals with different genetic types of polycystic kidney disease have illustrated that chemical and dietary inhibition of cyst growth initiated early during disease progression markedly slows the decline of renal function (Grantham, J. J., A. B. Chapman, and V. E. Torres, Volume progression in autosomal dominant polycystic kidney disease: the major factor determining clinical outcomes. Clin J Am Soc Nephrol, 2006. 1(1): p. 148-57). Total kidney volume (TKV) is an important biomarker for the assessment of the severity and the progression of ADPKD in humans, as it is also known to be associated with declining GFR (Grantham, J. J., et al., Volume progression in polycystic kidney disease. N Engl J Med, 2006. 354(20): p. 2122-30; Perrone, R. D., et al., Total Kidney Volume Is a Prognostic Biomarker of Renal Function Decline and Progression to End-Stage Renal Disease in Patients with Autosomal Dominant Polycystic Kidney Disease. Kidney Int Rep, 2017. 2(3): p. 442-450; Yu, A. S. L., et al., Baseline total kidney volume and the rate of kidney growth are associated with chronic kidney disease progression in Autosomal Dominant Polycystic Kidney Disease. Kidney Int, 2018. 93(3): p. 691-699).

The growth of kidney cysts in ADPKD is involved in multiple molecular pathways (Chebib, F. T. and V. E. Torres, Autosomal Dominant Polycystic Kidney Disease: Core Curriculum 2016. Am J Kidney Dis, 2016. 67(5): p. 792-810). Genetic mutations in ADPKD result in a reduction in intracellular calcium, which cascades to an increase in cyclic adenosine monophosphate (cAMP), activation of protein kinase A, and an increase in sensitivity of the collecting duct to the tonic effect of vasopressin. Vasopressin promotes fluid reabsorption and urine concentration by stimulating the formation of cAMP in cells of the renal collecting duct throughout the day. Blockade of vasopressin activity by the administration of vasopressin v2 receptor blockers or by simply increasing water intake has been shown to significantly retard the growth of renal cysts in rodents (Nagao, S., et al., Increased water intake decreases progression of polycystic kidney disease in the PCK rat. J Am Soc Nephrol, 2006. 17(8): p. 2220-7). Furthermore, abnormal epithelial chloride secretion occurs through the cAMP-dependent transporter, contributing to generating and maintaining fluid-filled cysts in ADPKD.

The relationship between kidney volume and function appear complex and multifactorial. For example, a substantial decline in TKV in association with improvement in renal function is observed in the native polycystic kidney in patients with ADPKD after renal transplantation (Yamamoto, T., et al., Kidney volume changes in patients with autosomal dominant polycystic kidney disease after renal transplantation. Transplantation, 2012. 93(8): p. 794-8; Jung, Y., et al., Volume regression of native polycystic kidneys after renal transplantation. Nephrol Dial Transplant, 2016. 31(1): p. 73-9). Although it is not clear which factors after transplantation influence the decrease in TKV, the greater reduction in TKV in the patients with better renal function after transplant may be related to more efficient elimination of uremia effects on proliferation of the tubular epithelium and greater reduction of blood flow to the native kidneys.

As a potential treatment method for ADPKD, the inventors developed a 3D mechanical device that is anatomically fitted to the kidney to inhibit the structural progression of the kidney. Without wishing to be bound to any specific theory, the inventors consider that an external mechanical constraint, applied to restrict the volumetric growth of the kidney, would generate hydraulic pressure competing against the transepithelial osmotic gradient that favors cyst growth and expansion. Subjecting polycystic kidneys to the structural constraint may lead to the suppression of the tonic effect of vasopressin, thereby forestalling progressive development and expansion of kidney cysts. Furthermore, the anatomical significance of space requirement for the expansion of polycystic kidney is evidenced by the fact that the right kidney tends to be smaller than the left kidney, since the former is more confined in space by the liver and surrounding organs than the latter in the abdominal cavity.

Configuration and Construction of Capsule Device

Imaging of Kidney for the Configuration of Capsular Device and Surgical Planning

Patients with ADPKD are highly variable in their kidney size and shape. Thus, it is critical and preferable to acquire high-resolution 3D images of the kidneys to design and manufacture the proposed kidney capsular device. The radiological imaging is also important for surgical planning for the implantation of the device. In order to precisely visualize the 3D anatomy of the kidney and surrounding structures, high-resolution 3D radiologic imaging modality such as MRI or CT is important. These imaging techniques are commonly used in a daily clinical setting: for example, for a living donor work-up for kidney transplantation. MRI or CT imaging provides a wealth of anatomical information essential for the pre-transplant evaluation, including the kidney morphology, number and size of renal vessels, aberrant anatomy, and surgical accessibility. Also important for the device design and surgical planning is the configuration of kidney cyst such as large exophytic cysts. Some exophytic cysts could be removed surgically prior to the placement of the capsular device to reduce the overall burden of cysts and improve the fitting of the device to the kidney. From pre-operative images, large exophytic cysts can be graphically excluded from images by means of image processing. The device, when configured according to the processed images and consistent with the surgical plan, would improve the anatomical fidelity between the device and kidney and the effectiveness of constraining the structural progression of ADPKD.

Certain embodiments of the present invention relate to a capsule device to encase the kidney. A capsule device to encase the kidney can be used to slow down or halt the abnormal growth of the kidney associated with diseases such as polycystic kidney disease. The capsule device to encase the kidney may be designed to cover substantially the entire kidney. The term “covering substantially the entire kidney” means covering a large part of the kidney volume: for example, to cover 70% or more, 80% or more, 90% or more, 95% or more, or 98% or more. The capsule device to encase the kidney is designed to suppress the increase of total kidney volume.

Anatomically, the renal artery, renal vein, and ureter are connected to the kidney. The capsule device encasing the kidney may be provided with an aperture so as to substantially cover the kidney without disturbing important anatomical structures such as the renal artery, renal vein and ureter connected to the kidney. In other word, the capsule device may be configured to have an aperture that allows passage of the structures connected to the body organ. The aperture provided in the capsule device encasing the kidney may be configured to have individualized positions and sizes according to the size and shape of the patient's kidney to which the capsule device is implanted. For designing capsule devices wrapping individualized kidneys, medical imaging data of patients, including MRI, CT, ultrasound images, fluoroscopic images, and laparoscopic images, can be used. The capsule devices to encase individualized kidneys can be manufactured using three-dimensional manufacturing techniques such as automated 3D printing technology or manual manufacturing technology. For 3D printing, for example, CONNEX 3 OBJET 500 made by Stratasys, may be used. Some capsule devices may be designed on the basis of general size and shape of kidney without being personalized.

Materials and Fabrication of the Capsular Device

From the radiology images of the abdomen, kidney regions are segmented from surrounding visceral organs. The image segmentation process can be performed manually by an expert delineating the kidney border on each slice or semi- or fully-automatically by a computer program on a set of images. The size and shape of the device is designed to encase each segmented kidney region. If large exophytic cysts are to be removed prior to the placement of the capsular device, these cysts are graphically excluded from the kidney region that becomes the base model of the device. In addition, the capsular device is designed to contain anatomically appropriated apertures or gaps through which anatomical structures such as blood vessels, collecting system, and ureters connected to the kidney are unhindered by the device while containing the remaining body of the kidney. The capsule device is configured precisely with personalized anatomical apertures to ensure that key anatomical structures including blood vessels, ducts, and tubes connected to the body organ are not interfered with from the main volume of the organ encased by the device.

Using the processed 3D imaging model of the segmented kidney capsule, a device is fabricated using 3D printing techniques or manually by a skilled expert in device configuration. The material properties and thickness of the device is determined in consideration of the growth projection of the kidney including the patient's age, gender, allergic sensitivity profile, current kidney size, kidney cyst pattern, ADPKD classification, demographic and clinical information. For example, patients with projected rapid growth of the kidney may require a more rigid and thicker capsule that exerts a greater force to constrain the growth than those with projected slow growth of the kidney. The choice of the material includes silicone rubbers (silicone elastomers that have been widely used in medical product applications due to biocompatibility, superior temperature and chemical resistance, good mechanical and electrical properties, and natural clarity). Other materials that may be used include natural rubber, urethane resin, cellulose, polytetrafluoroethylene resin, fibers such as silk and polyester, and proteins such as collagen. Alternative to silicone-like elastic materials, rigid materials such as surgical mesh may be used to wrap around and constrain the growth of body organs or mass. Basic materials used for surgical mesh include synthetic polymers, biological acellular collagen, and composite materials. Moreover, instead of using only a single type of material or configuration, a combination of elastic and rigid materials in juxtaposed configurations such as sandwich layers of silicon rubber and surgical mesh may be used. The composite materials and configurations may provide balanced tensile and compressive strength required for maintaining the structural integrity and surgical closure of capsule while constraining body organs or mass. Appropriate materials should have superior compatibility with human tissue and fluid, extremely low tissue response when implanted, maintain sterility, tolerability in a wider range of temperature extremes, high tear and tensile strength, good elongation and flexibility. For silicon rubbers (silicon elastomers), there are various types of fabrication methods including extrusion, injection molding, compression molding, transfer molding, blow molding, rotational molding, vacuum or thermoforming, matched molding, and low-pressure molding. Using the 3D kidney structure from the radiology images as a template and mold, the appropriate molding and fabrication process is selected with considerations of clinical and final product design criteria.

Some aspects of the present invention relate to a method of manufacturing a capsule device to encase a body organ, comprising of measuring the shape of a body organ of a patient; designing a capsule device adapted for the body organ; and fabricating the capsule device. The patient may be an animal, particularly a mammal and more particularly a human. Exemplary body organs include kidney, liver, and ovary. The measuring can be performed using MRI, CT, ultrasound images, fluoroscopic images, or laparoscopic images. The fabrication can be performed using three-dimensional manufacturing techniques such as automated 3D printing technology or manual manufacturing technology. The designing may comprise determining bio-compatible material, elastic property, configuration, and/or size of the capsule device based on individual subject's medical information selected from the group consisting of: subject's age, subject's gender, subject's allergic sensitivity profile, target organ's anatomy, and target organ's projected growth rate. For instance, the capsule device may have a shape approximating to the target body organ. The designing may comprise including predetermined surgical opening and closing suture lines within the device by consideration of efficient surgical procedures for the implantation of the device. The designing may comprise including a means to close the split openings of the device. The mechanism or means for closing the split sections of the device may be selected from the group consisting of interlacing strings, buttons, hooks, fasteners, hook-and-loop fasteners or other closing mechanisms crisscrossing the device's split openings. The designing may include broadening the edges of the device outward, for example at right angles, along the circumference of split openings such that the broadened, opposite facing edges juxtapose and overlap each other. The overlapping edges are harnessed, for example, by suturing, threading, or gluing to reinforce the closure of split openings. The designing may comprise selecting liquid or flexible injectable material for implanting and fabricating the capsule devices via minimally-invasive or laparoscopic surgical procedures.

Design and Packaging of the Capsule Device to Facilitate Surgical Implantation

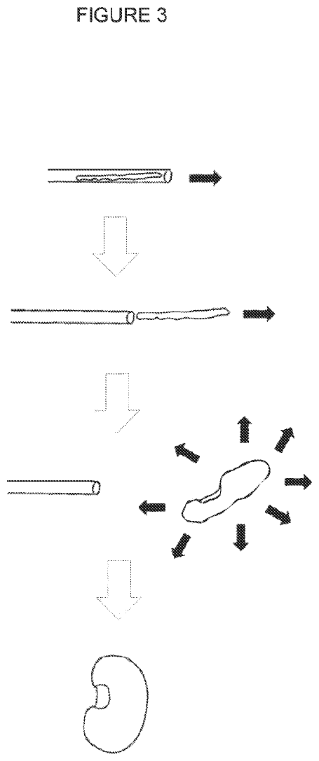

There are two key surgical steps required in the implantation of the capsule device to kidney: the access to the target kidney; and the encasement of the target kidney with the capsule device. The target kidney can be surgically accessed via open surgical or minimally-invasive laparoscopic surgical procedure. In general, the laparoscopic procedure is preferred over the open surgery because it requires smaller incisions and leads to a faster recovery. However, the laparoscopic procedure is technically more demanding and may not be clinically appropriate in some patients. Compared to the open surgery, the laparoscopic surgical procedure is more restricted in space for the access and delivery of the capsule device to the target kidney. In order to introduce the capsule device to the perirenal space via small surgical incisions, the capsule device could be tightly folded and packaged in a catheter that is inserted into the body cavity (FIG. 3). When the catheter is accessed to the perirenal space, the delivered capsule device is released from the catheter. Subsequently, the deployed capsule device is unfolded and expanded within the perirenal space prior to the implantation over the kidney. An alternative form of the capsule device may be made of liquid or flexible injectable material that is injected via a catheter into the perirenal space to envelope over the kidney surface. Once the enveloped material is solidified like a screen layer, it could exert a mechanical constraint to inhibit the expansion of the kidney out of the perirenal space.

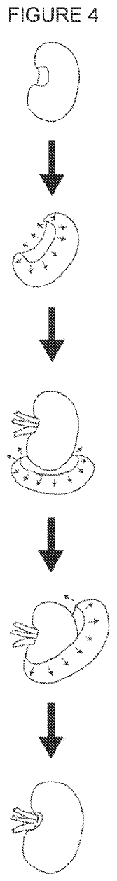

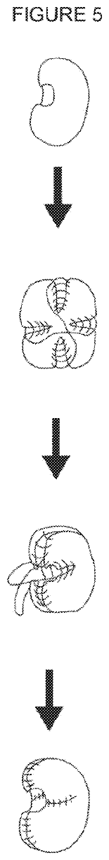

After the capsule device is delivered to the perirenal space of the target kidney via open or laparoscopic surgical procedure, the encasement of the entire volume of the target kidney by the capsular device is performed. A capsule device that is elastic and stretchable may be manually stretched over its opening to wrap around the kidney (FIG. 4). When the elastic opening of the capsule device is sufficiently enlarged to place and encompass one pole, the lower or upper pole, of the kidney, the device can be subsequently mobilized and stretched further to encase the entire kidney volume within the capsule device. When the capsule device is rigid or not stretchable over the entire kidney, it is preferred to split open or configure the device in sections to facilitate the segmental wrapping of the kidney. The split sections need to be juxtaposed and sealed following the encasement of the kidney to mechanically constrain the growth of the entire kidney. The efficiency of closing of the split sections can be improved by inclusion of predetermined surgical opening and closing suture lines within the device. The split sections may have broadened edges that overlap each other and are harnessed to reinforce closure. Following the wrapping of all sections of the kidney by the capsule device, the split open lines of the device are closed to complete the encasing by means of pulling interlacing strings, buttons, hooks, fasters, hook-and-loop fasters, or other closing mechanisms crisscrossing the open lines (FIG. 5). Adhesives or fusion by heat may be used to close the split sections. Improved efficiency in encasing the kidney and closing the sectionalized capsule device would lessen surgical time and technical complexity. The capsule device may be formed of fabric (cloth, fiber), for example, having high, modest, or low stretchability.

In some embodiments of the present invention, as mentioned above, the capsule device may be configured to include predetermined surgical opening and closing suture lines within the device by consideration of surgical procedures for the implantation of the capsule device. For example, the capsule device may be designed to be split open at the placement to wrap the kidney. The capsule device to encase the kidney may be designed to be closed after completion of wrapping by using crossed strings, buttons, hooks, fasteners, hook-and-loop fasteners, or other closing mechanisms that intersect the split openings. The capsule device to encase the kidney may be made of liquid or flexible injectable material capable of being implanted by minimally-invasive or laparoscopic surgical procedures. The injectable material is capable of being implanted by minimally-invasive or laparoscopic surgical procedures. In some embodiments of the invention, the capsule device may be designed to be closed after completion of wrapping by using a closing means including crossed strings, buttons, hooks, fasteners, and hook-and-loop fasteners.

Configuration of the Capsule Device to Facilitate Monitoring after Placement

After the placement of the capsule device, the size and growth of the kidney as well as the structural change of the capsule could be evaluated and monitored by means of follow-up quantitative imaging. In order to facilitate the assessment of structural changes, fiducial markers, that are geometrically aligned and visualized on the acquired images, are stitched or embedded in the capsule device. Alternatively, the existing structures serving as the closing mechanisms of the device opening, such as interlacing strings, buttons, and hooks that crisscross the device's split openings, could be used as fiducial markers. Although radio-opaque metallic markers are commonly used for x-ray imaging markers, they may cause artifacts and negatively affect the image quality in CT or MRI images. For CT, iodine-based radio-opaque materials could be used for fiducial markers. In addition to iodine, materials that are of high x-ray attenuation to image and document structural changes of the capsule could be used for fiducial markers for CT imaging. For MRI, gadolinium- or manganese-based materials that have been used clinically to image and enhance MR contrast could be used for fiducial markers. Monitoring changes in the position of fiducial markers and in the size and shape of kidney with serial imaging studies would provide important information for the evaluation of therapeutic efficacy of the capsule device and improve clinical management of patients with ADPKD who are treated with the capsule device.

Placement of the Capsule Device

Perirenal Anatomical Spaces

The abdominal cavity is subdivided into two parts: the anterior part (the peritoneal space) and the posterior part (the retroperitoneal space). The retroperitoneal space includes the perirenal space containing kidneys and adjacent pararenal structures such as the renal hilar vessels, renal pelvis and proximal ureter, adrenal glands, and perirenal fat. These contents are enclosed between the renal fascial layers. The kidney is enclosed in an envelope of perirenal fat, which in turn is sheathed within the renal fascia. The perirenal space is an inverted cone of tissue that lies lateral to the lumbar spine and is confined by the anterior renal fascia and posterior renal fascia. These fasciae are dense elastic connective tissue sheaths less than 2 mm thick. These fasciae define the anterior and posterior borders of the perirenal space. The perirenal space contains numerous thin septations. The medial perirenal spaces communicate with the great vessel space by way of the hilar vessels. The anterior portion of the retroperitoneum anterior to the perirenal space is called the anterior pararenal space, while the posterior portion of the retroperitoneum posterior to the perirenal space is called the posterior pararenal space.

Approaches to the Surgical Implantation of the Capsule Device

The capsule device could be placed via either an open or laparoscopic surgical approach. The open surgical approach could be transperitoneal or retroperitoneal, with the latter preferred. The laparoscopic surgical approach could be transperitoneal or retroperitoneal and could be robotic or hand-assisted. Although the laparoscopic surgical approach is less invasive than the open approach, it may involve more complex surgical maneuvers.

Pre-operative abdominal CT or MR imaging studies including CT angiography or MR angiography are essential for surgical planning. The evaluation of the overall abdominal anatomy, kidney morphology, renal vessels, collecting system and ureter will provide a guide for the access to the target kidney and the approach to the implantation of the capsule device. The aorta and inferior vena cava contribute the main inflow and outflow vessels to the kidneys, respectively. Although each kidney is usually fed by a single renal artery and vein, anatomic variants of the renal vasculature is common. The position, numbers, and presence of accessory vessels must be carefully evaluated for surgical planning.

The kidney can be surgically accessed through two separate planes: the anterior pararenal space and the posterior pararenal space. The anterior pararenal space is often empty and best approached by incising the posterior parietal peritoneum through the anterior renal fascia. The incision of the medial anterior renal fascia allows access to the renal hilum and colonic mobilization. The posterior pararenal space is usually filled with fat and is developed by dissecting between the posterior renal fascia and the transversalis fascia lining the posterior abdominal wall. The dissection of the medial aspect of the posterior renal fascia leads to the renal hilum through the anterior. Some patients with ADPKD may require progressive cyst aspiration and decortication to facilitate the identification of vital structures and the creation of enough abdominal cavity space to operate, especially for laparoscopic approach.

The choice of surgical approach and skin incision depends on a number of factors such as the location of the kidney, patient's body habitus, and physician's preference. Commonly used incisions include the flank, thoracoabdominal, and transabdominal incisions. In the flank approach, the pararenal space is developed by dissecting the posterior layer of the renal fascia from the muscles of the posterior abdominal wall. Subsequently, the dissection of the anterior layer of the renal fascia away from the peritoneum and colonic mesentery exposes the fascial compartment containing the kidney, adrenal gland and perirenal fat. The perirenal fat is pushed away from the kidney using dissection and electrocautery. Further separation of the adrenal gland from the upper pole of the kidney and the superior attachments of the kidney to the spleen, pancreas, and liver allow safe caudal retraction of the kidney. After the mobilization of the lower pole of the kidney, the ureter can be identified and separated on the peritoneal side of the incision. The posterior lateral retraction of the kidney allows the visualization of the renal hilum medially. In the thoracoabdominal approach, an incision over the rib begins at the posterior axillary line medially across the costal cartilage margin to the midline and then carried down the midline to the umbilicus. The pararenal space is developed by dissecting the abdominal wall muscles into the peritoneum. The pleural space is also entered including the division of the diaphragm. In the transabdominal approach, an incision is made below the costal margin and extended medially to the xiphoid process and then across the midline. The peritoneal cavity is entered by dissecting the abdominal wall muscles.

Irrespective of the choice of surgical approach, after the development of the pararenal space and fascial compartment, a careful separation of perirenal fat and adrenal gland is crucial to expose the kidney for the placement of the capsule device. After the kidney is accessed and mobilized, the device is placed over the kidney. With the completion of the device placement, the main volume of the kidney is encased by the device, while the renal hilar structures including the collecting system, ureter, renal arteries and veins are unhindered and pass through the aperture of the device.

Consideration in Capsule Device Design and Placement for Non-Renal Organs

The design and configuration of the capsule device would require the accurate assessment of morphology and disease progression involved in the organ of interest. For this purpose, 3D medical imaging modality such as MRI, CT, and ultrasound imaging can be used to image the anatomy of the organ and adjacent body structures. After the relevant images are obtained, the region representing the target organ or mass to be constrained by the capsule device is segmented from surrounding anatomical structures. For example, for the treatment of polycystic liver disease, the entire liver or a portion of the liver could be a target region. The target liver region is manually delineated or automatedly segmented from the images in consideration of the adjacent structures including critical vascular and ductal structures such as the hepatic artery, portal vein, hepatic vein, and bile duct. The neighboring structures including diaphragm, right kidney, spine, duodenum, stomach and right colon should also be considered in the segmentation of the target liver region as well as in planning for the placement of the manufactured capsule device. Because of the larger size and more complex anatomy, the capsule device for the liver is likely more complicated and difficult than that for the kidney in designing, manufacturing, and placement. In addition, the selection of the capsule device material and thickness should be determined by factoring in the growth projection of the liver and affected cystic volume. Additional variables that may be considered include the patient's age, gender, allergic sensitivity profile, and demographic and clinical information.

Method for Treatment and Prevention of Diseases Accompanying Abnormal Growth of a Body Organ or Mass

Certain aspects of the present invention relate to a method for treating and preventing a disease associated with abnormal growth of a body organ or mass, comprising the implantation of a capsule device to encapsulate a patient's body organ or mass. The patient may be an animal, particularly a mammal and more particularly a human. Exemplary body organs include kidney, liver, and ovary. The diseases associated with abnormal growth of a body organ may be, for example, diseases of the kidney, liver, or ovary, and the diseases of the kidney may be, for example, polycystic kidney disease, especially ADPKD or ARPKD. In some embodiments of the present invention, the method for treating and preventing a disease associated with abnormal growth of a body organ or mass may further include at least one of: measuring the body organ or mass, designing the capsule device, manufacturing or selecting the capsule device, and monitoring the implanted capsule device.

Use of Capsule Devices

Some embodiments of the present invention relate to a use of the capsule device to encase a body organ such as kidney, liver and ovary. The patient may be an animal, particularly a mammal and more particularly a human. Exemplary body organs include kidney, liver, and ovary. The diseases associated with abnormal growth of a body organ may be, for example, diseases of the kidney, liver, or ovary, and the diseases of the kidney may be, for example, polycystic kidney disease, especially ADPKD or ARPKD.

Specific examples of the present invention are illustrated below, but the present invention should not be construed as being limited by these examples.

### EXAMPLES

### Example 1: Animals

Han:SPRD-Cy/+ rats with polycystic kidneys were bred and maintained as previously described at the Education and Research Facility of Animal Models for Human Diseases at Fujita Health University (Yu, A. S. L, et al., Baseline total kidney volume and the rate of kidney growth are associated with chronic kidney disease progression in Autosomal Dominant Polycystic Kidney Disease. Kidney Int, 2018. 93(3): p. 691-699; Nagao, S., et al., increased water intake decreases progression of polycystic kidney disease in the PCK rat. J Am Soc Nephrol, 2006. 17(8): p. 2220-7). The protocols for the use of these rats were approved by the Animal Care and Use Committee of Fujita Health University.

The breeding of male and female heterozygote (Cy/+) rats produced littermates containing Cy/+, Cy/Cy, or +/+ genotypes. Cy/Cy animals with three weeks lifespan were not used for the study. To select Cy/+ animals used for the current study, mutational analysis revealed a C-to-T transition in Pkdr1 (Anks6), the responsible gene name of Cy. At 4 weeks of age, +/+ and Cy/+ rats were distinguished using a PCR-RFLP method as described in a previous study (Brown, J. H., et al. Missense mutation in sterile alpha motif of novel protein SamCystin is associated with polycystic kidney disease in (cy/+) rat. J Am Soc Nephrol 16, 3517-3526 (2005)). PCR products were obtained using the Applied Biosystems GeneAmp PCR System 9700. Wild-type Sprague-Dawley (SD) rats with normal kidneys were purchased from Charles River Japan (Kanagawa, Japan) at 4 weeks of age.

### Example 2: Fabrication of Capsule Devices

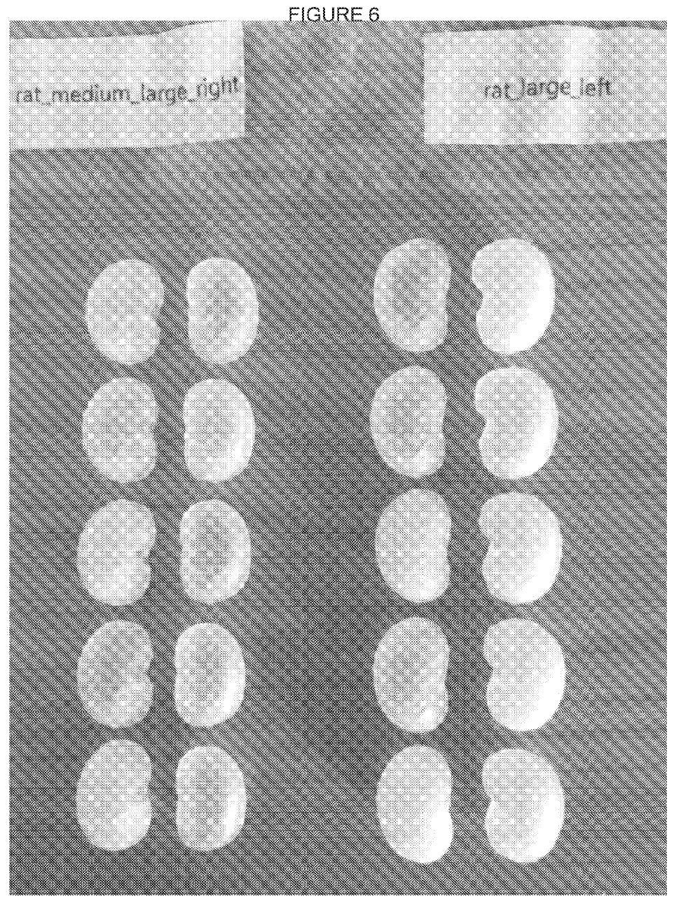

A wild-type rat at 28 weeks old and a Han:SPRD-Cy/+ PKD rat at 22 weeks old were anesthetized and scanned using the R-mCT2 microcomputed tomography scanner (Rigaku Corporation, Tokyo, Japan). Following the administration of iodinated contrast medium, CT images of the abdomen were acquired. From each CT image set, the right and left kidney regions were identified and segmented semi-automatically using an in-house image segmentation program. The segmented kidney regions from the wild-type rat provided the small template, while the segmented kidney regions from the PKD rat corresponded to the large template. Using these two templates, three intermediate-size templates were generated by mathematical interpolation of 3D volumetric data points between the small (i.e., zero percentile) and large templates (i.e., 100 percentile): the medium template corresponding to the 50 percentile, the small-to-medium template corresponding to the 25 percentile, and the medium-to-large corresponding to the 75 percentile of the volumetric data (FIG. 6). From the five different-sized templates, 3D volumetric models of the rat kidneys were produced. The dimension of the 3D voxel was isotropic 0.15×0.15×0.15 mm. Each 3D volumetric model was designed to encase the entire volume of each segmented kidney region. Furthermore, the 3D volumetric model was configured to contain an aperture at the renal hilar region, through which hilar structures such as blood vessels, collecting system, and ureters connected to the kidney were able to pass unhindered while the remaining body of the kidney was encased.

Using the processed 3D volumetric models of the segmented kidney regions, kidney capsule devices were fabricated using 3D printing technique (CONNEX 3 OBJET500, Stratasys, MN, USA). The 3D printer jetted a liquid photopolymer onto a build tray and cured it with ultraviolet light (Mitsouras, D., et al. Medical 3D Printing for the Radiologist. RadioGraphics 35, 1965-1988 (2015)). Two jetting heads sprayed layers of the part: one set for the kidney capsule device material, and one set for support material. The tray was incrementally lowered layer by layer to print a 3D structure. Because the kidney capsule device was a shell-like structure with a complex geometry, the printer required a support material (SUP705, Object, Inc., MA, USA) that upheld overhangs and filled the inside of the empty space of the capsule device. Following the completion of the printing, the supporting material was removed by using pressurized water sprays to uncover the kidney capsule device. For the 3D printing material of the capsule device, we used a durable rubber-like photopolymer that can stand up to repeated flexing and bending (Agilus30 Stratasys, MN, USA). This material was well suited to facilitate the surgical placement of the capsule device for the purpose of encasing rat kidneys in vivo.

### Example 3: Transplantation of Capsule Devices to Animals

A total of six surgical sessions were held over the period of 14 months using seven wild-type rats with the age range of 7-8 weeks old and six Han:SPRD-Cy/+ PKD rats with the age range of 6.5 weeks old. In the first session of the experimental series, mainly for the purpose of testing the feasibility of the surgical implantation of the capsule device, the left kidney of a wild-type SD rat at 8 weeks of age was encased with the small capsule. The rat was anesthetized with three types of mixed anesthetic agent prepared with 0.375 mg/kg of medetomidine, 2.0 mg/kg of midazolam, and 2.5 mg/kg of butorphanol. The left flank of the rat was prepped and dissected to access the retroperitoneal and perirenal space. The left kidney was identified and fully exposed after carefully separating the perirenal fat tissue. The small capsule device was cut and split transversely approximately a third to a half of the circumference starting from the aperture. Along the cut plane, the capsule device was gently split in the middle and bent backward to open the lower pole of the device. The exposed kidney was drawn into the device in the lower pole. After the lower pole of the kidney was placed, the device was carefully stretched and pulled over the remaining kidney body. The entire kidney volume was encased by the capsule device, while the renal hilar structures were uncovered and unhindered within the hilar aperture of the device. The split line of the device was sutured and sealed by application of surgical glue. The capsuled kidney was covered by the perirenal tissues and retroperitoneum. The dissected retroperitoneal fascia and skin were sutured. During this session, another wild-type rat underwent a sham operation without implantation of capsule device. After the follow-up of 5.4-5.6 weeks, rats were sacrificed to retrieve both the capsuled and uncapsuled kidneys. Blood samples were collected.

In the second session, the left kidney of a Cy/+ PKD rat at 6.5 weeks of age was implanted with the medium-sized capsule device. The medium-sized capsule device was chosen because this size was deemed appropriate. The small and small-to-medium capsules appeared too small to fit to the kidney of PKD rats. The surgical procedure was identical to that of the first session. This rat was followed for 7.1 weeks and sacrificed to retrieve both the capsuled and uncapsuled kidneys.

In the third session, two wild-type SD rats at 6.5 weeks of age from the same set of siblings underwent surgery: the first rat for bilateral sham operation and the second rat for the implantation of the medium-sized capsule device in the right kidney. The surgical procedure was identical to that of the previous sessions. Both rats were followed for 12 weeks and sacrificed to retrieve both the capsuled and uncapsuled kidneys.

In the fourth session, three wild-type SD rats at 7 weeks of age from the same set of siblings underwent surgery: the first rat for bilateral sham operation, the second rat for the implantation of the medium-sized capsule device in the left kidney, and the third rat for the implantation of the medium-sized capsule device in the right kidney. The surgical procedure was identical to that of the previous sessions. All three rats were followed for 12 weeks and sacrificed to retrieve both the capsuled and uncapsuled kidneys.

In the fifth session, three Cy/+ PKD rats at 6.5 weeks of age from the same set of siblings underwent surgery: the first rat for bilateral sham operation, the second rat for the implantation of the large-sized capsule device in the left kidney, and the third rat for the implantation of the large-sized capsule devices in the bilateral kidneys. The large-sized capsule device was chosen because these PKD rats were heavier than the PKD rat used in the second session. The surgical procedure was identical to that of the first and second sessions for the rat with the implantation of the capsule device in the left kidney only. The surgical procedures for the other two rats were involved in the bilateral kidneys. All three rats were followed for 12 weeks and sacrificed to retrieve both the capsuled and uncapsuled kidneys.

In the sixth session, two Cy/+ PKD rats at 6.5 weeks of age from the same set of siblings underwent surgery: the first rat for bilateral sham operation and the second rat for the implantation of the large-sized capsule device in the left kidney. The surgical procedure was identical to that of the previous sessions. Both rats were followed for 12 weeks and sacrificed to retrieve both the capsuled and uncapsuled kidneys.

### Example 4: Effects of Transplantation of Capsule Devices

To investigate the effectiveness of a mechanoanatomical method for halting the progression of ADPKD, the inventors used the Han:SPRD-Cy/+(Cy/+) rat model, which develops polycystic kidney disease. The 3D capsule interventional devices were designed and constructed to encase rat kidneys. Micro-computed tomography images acquired from a wild-type rat at 28 weeks old and a Cy/+ rat at 22 weeks old were used to provide the anatomy reference templates for the configuration of the 3D kidney capsules. With mathematical interpolation of the anatomy reference templates, 3D imaging models of the rat kidney capsules were generated at five different sizes (small, small-to-medium, medium, medium-large, large). The 3D imaging models were input into a 3D printer to produce the kidney capsule devices to encase rat kidneys in vivo (FIG. 6).

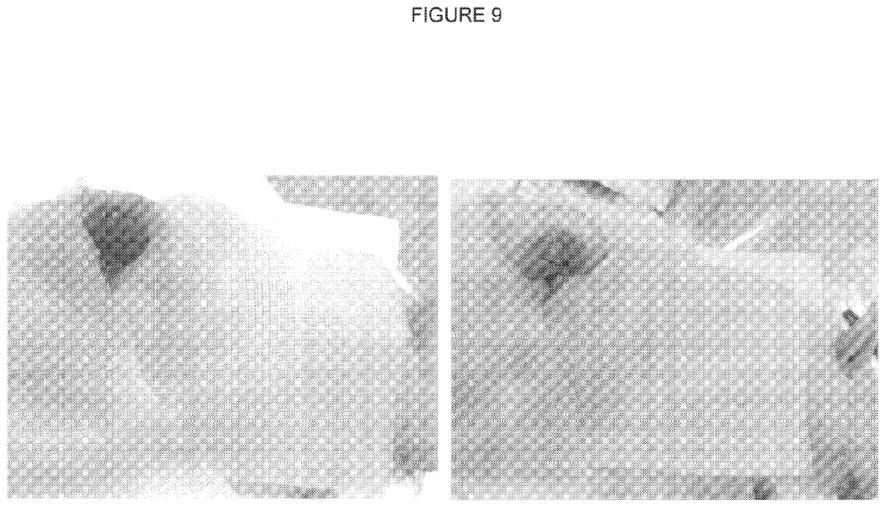

The capsule devices were surgically implanted to kidneys of wild-type and Cy/+ rats (FIG. 9). Rats were haphazardly determined for unilateral or bilateral implantation of capsule devices. Sham operations were also performed on both wild-type and Cy/+ rats. The rats tolerated the surgical procedures well and recovered, and they were followed afterwards. After follow-up periods that were deemed sufficiently long for the growth of operated kidneys and determined conveniently for surgical schedule, rats were sacrificed to retrieve the kidneys and capsules. The results from the individual rats at different sessions are summarized in Table 1. Consistently significant reductions in kidney size were noted in the Cy/+ kidneys encased by the capsule devices, compared to the kidneys that were not encapsulated.

In the wild-type rats, the observed change in kidney size between the capsuled and uncapsuled kidneys was slight. This is most likely because the kidneys of these wild-type did not grow beyond the volumetric capacity of the implanted small-size capsules during the follow-up period. The small-sized capsule device implanted inside the wild-type rats was constructed based on the micro-CT images of a 28-week-old wild-type rat, while the capsuled wild-type rats at the time of autopsy were 13.4-13.6 weeks old. If the follow-up of the wild-type rats had been extended beyond the age of 28 weeks at autopsy, we may have observed the effect of the capsule device limiting the normal growth of the wild-type kidneys for differentiating the size between the capsuled and uncapsuled kidneys.

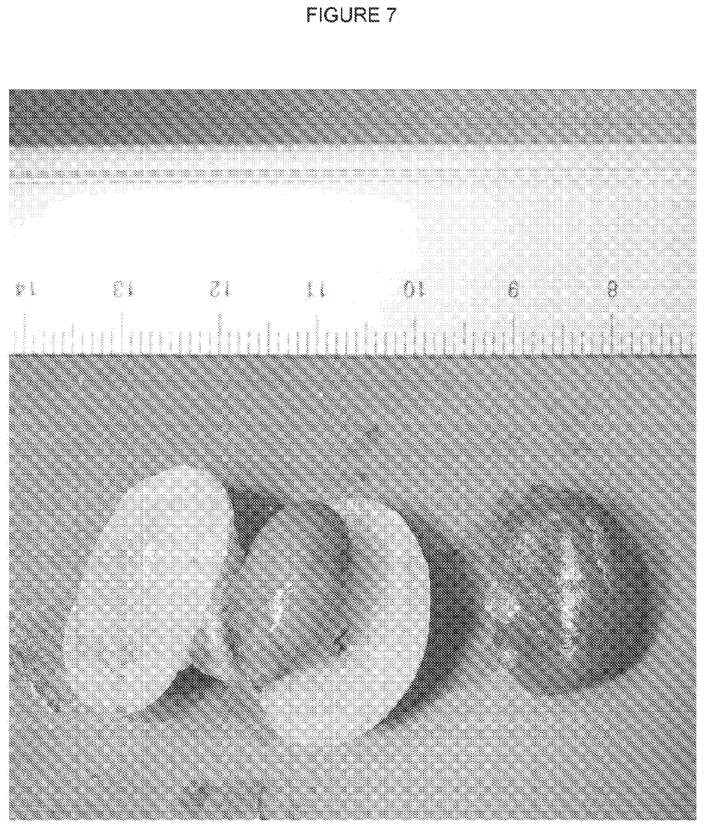

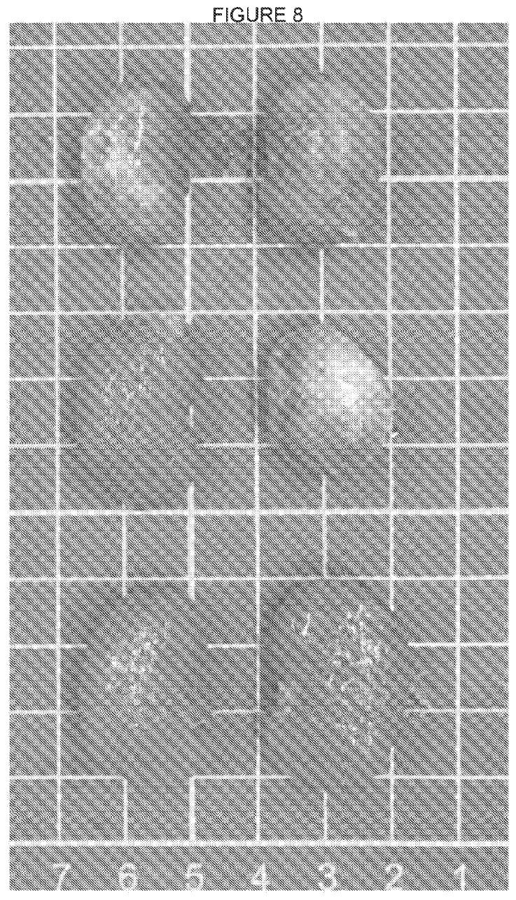

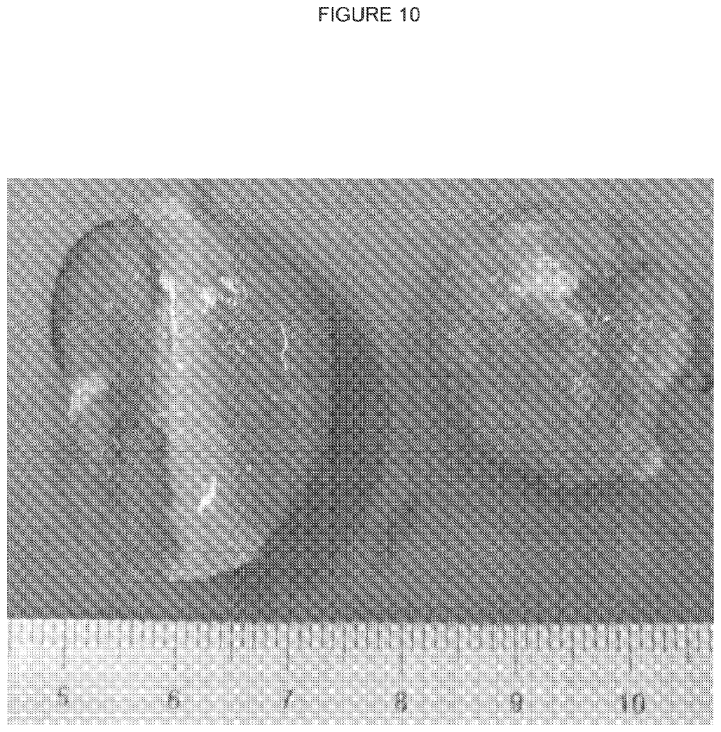

In PKD rats, the sizes of the capsuled kidneys were consistently and significantly smaller (reduction by 21-36%) than those of the uncapsuled kidneys (FIGS. 7, 8 and 10). There were some variations in the kidney size growth among the PKD rats. For example, the PKD rat in the second session was greater in absolute kidney size (uncapsuled: 6.14 g, capsuled: 3.91 g) despite a shorter follow-up period (7.1 weeks) than those in the third session (uncapsuled: 4.23-4.70 g, capsuled: 2.79-3.73 g, follow-up period 12 weeks). This could be related to individual variations in rats, as the three rats in the third session from the same set of siblings were of similar kidney size distribution.

Capsule devices of different sizes were implanted depending on the genotype and weight of rats. Additional factors that need to be considered for the capsule device are the material properties and thickness of the device as well as the growth projection of the kidney to be constrained. For example, kidneys with rapid projected growth may require more rigid and thicker capsules to achieve a greater mechanical force to constrain the growth than those with slow projected growth.

### Example 5: Histological Evaluation of Kidney

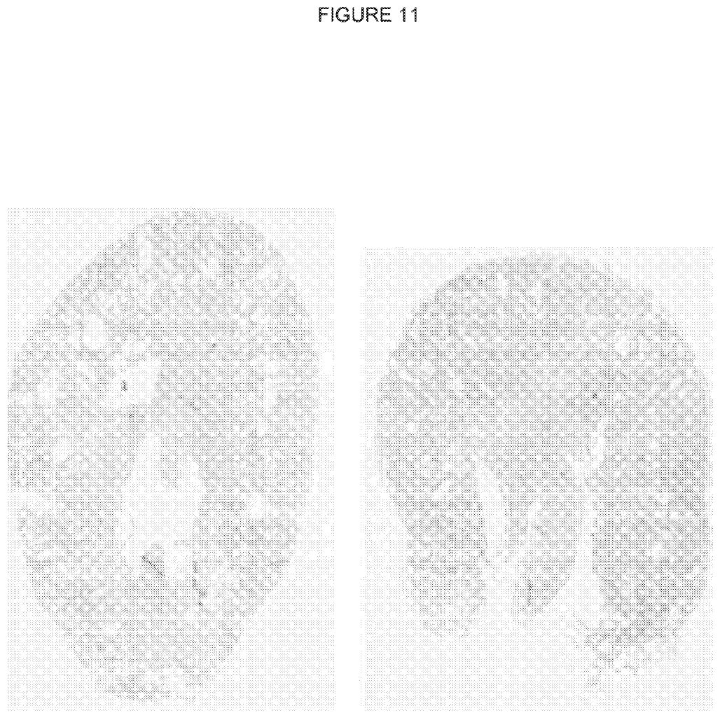

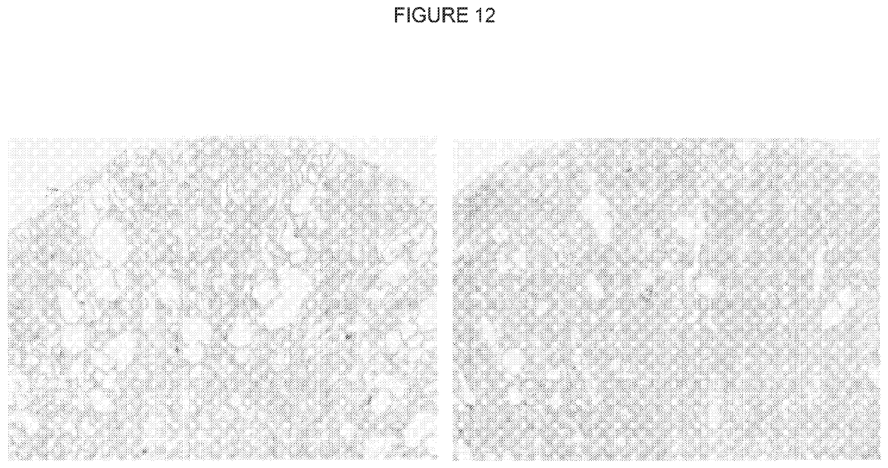

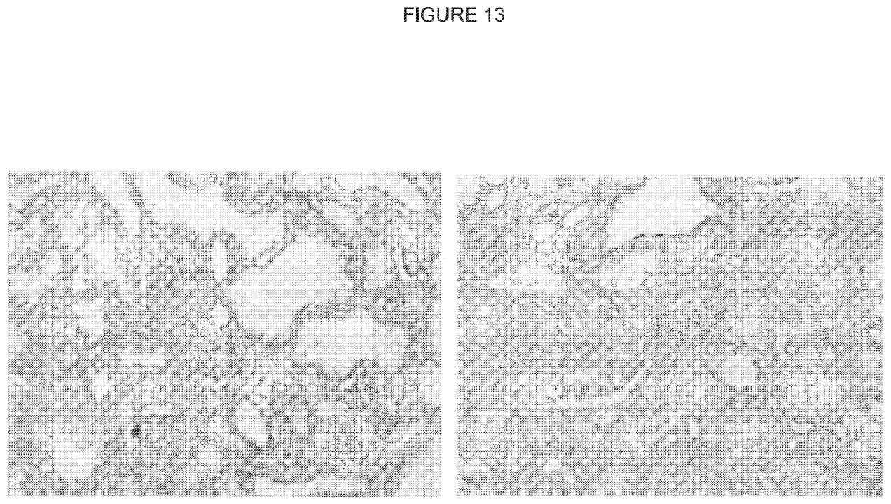

Histological evaluations of kidneys retrieved after operations on wild-type rats and Cy/+ rats are shown in FIGS. 11-14. In the wild-type rat with the capsule device implanted in the left kidney, the uncapsuled right and capsuled left kidneys showed no visible histological differences, particularly no dynamic inflammatory cell infiltration. In Cy/+ rats with the sham operation and capsule implantations, all the kidneys showed numerous renal cysts throughout renal parenchyma. However, the cysts in the capsuled kidneys were smaller in size than those of the uncapsuled kidneys, likely reflecting the differences in the overall kidney size (FIGS. 11 and 13). In addition, there were considerable thickenings in the intrinsic anatomical renal capsules of the kidneys implanted with the capsule devices, compared to the intrinsic anatomical renal capsules of the kidneys without the capsule devices (FIG. 12). The thickened intrinsic anatomical renal capsules in the histological evaluation seem to correspond with fibrotic changes. The inventors believe that the mechanical constraint or inflammatory change elicited by the presence of the capsule device led to reactive fibrotic changes in the native renal capsule.

Histological specimens from all the kidneys of Cy/+ rats demonstrated the presence of numerous mononuclear cells interspersed between renal cysts. The kidneys without the capsule devices contained more and larger renal cysts, more mononuclear cells, and more stimulated cellular proliferation than the kidneys with the capsule devices. In addition, the ratio of non-cystic renal tissue was higher in the kidneys with the capsule devices than those without the capsule devices.

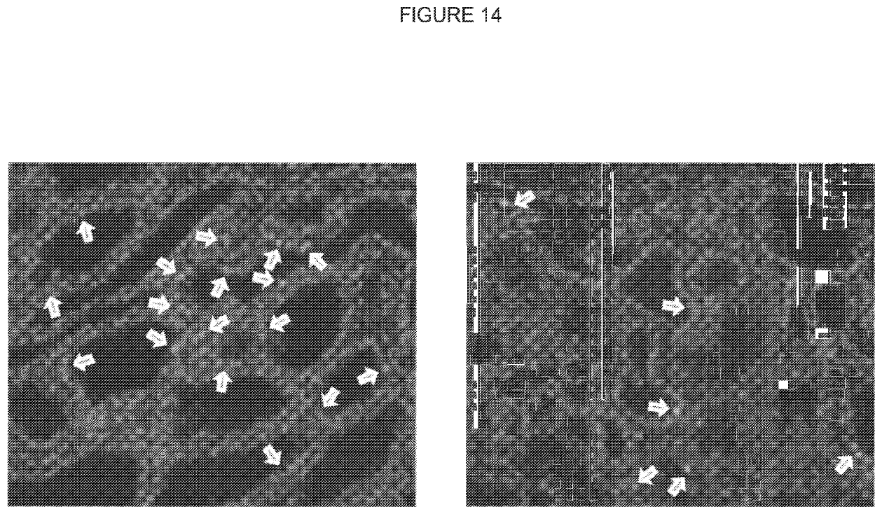

In the renal epithelial cells of cystic tubules and normal-like renal tubules of Cy/+ rats, levels of cell proliferation markers Ki67 and phospho-ERK were decreased in the capsuled kidneys compared to those in the uncapsuled kidneys (FIG. 14). CCR7-positive mononuclear cells were classified as M1 macrophages, which have roles in causing tissue damage. M1 macrophages were present mainly in cystic epithelia and were reduced in capsuled Cy/+ kidneys compared to the uncapsuled Cy/+ kidneys. For the wild-type rats, capsuled and uncapsuled kidneys showed no substantial differences in cell proliferation markers Ki67 and phospho-ERK.

Serum creatinine levels in Cy/+ rats with capsuled kidneys were consistently lower than those that underwent sham operation (Table 1). Table 1 below shows profiles and outcomes from Cy/+ rats at 3 different experimental sessions.

TABLE 1

| | | | | | | Body | Right | Left | Change % | Right | Left | Change % | | Change |
|---|---|---|---|---|---|---|---|---|---|---|---|---|---|---|
| | | Age at | Body | | Follow- | wt at | kid. | kid. | kidney wt | cyst | cyst | cyst area | | % Cr. |
| | | surgery | wt | Capsuled | up | autopsy | wt | wt | (capsule/ | area | area | (capsule/ | Cr. | (sham- |
| | Rat | (week) | (g) | kidney | (week) | (g) | (g) | (g) | uncaps.) | (%) | (%) | uncaps.) | (mg/dL) | surgery) |
| 2nd | #3 | 6.5 | 198 | Left | 7.1 | 503 | 6.14 | 3.91 | −36 | 50 | 35 | −30 | 0.63 | — |
| 5th | #9 | 6.5 | 238 | Sham | 12.0 | 516 | 4.58 | 4.70 | — | 41 | 55 | — | 0.91 | — |
| | #10 | 6.5 | 234 | Left | 12.0 | 486 | 4.23 | 2.79 | −34 | 40 | 26 | −34 | 0.74 | −19 |
| | #11 | 6.5 | 231 | Bilateral | 12.0 | 493 | 3.32 | 3.73 | −28, −21 | 26 | 23 | −36, −58 | 0.60 | −34 |
| 6th | #12 | 6.5 | 191 | Sham | 12.0 | 382 | 6.46 | 6.49 | — | 47 | 56 | — | 1.31 | — |
| | #13 | 6.5 | 218 | Left | 12.4 | 407 | 6.05 | 4.44 | −27 | 51 | 37 | −28 | 1.02 | −22 |

The present specification shows the preferred embodiments of the present invention, and it is clear to those skilled in the art that such embodiments are provided simply for the purpose of exemplification. A skilled artisan may be able to make various transformations and add modifications and substitutions without deviating from the present invention. It should be understood that the various alternative embodiments of invention described in the present specification may be used when practicing the present invention. Further, the contents described in all publications referred to in the present specification, including patents and patent application documents, should be construed as being incorporated the same as the contents clearly written in the present specification by their citation.

### INDUSTRIAL APPLICABILITY

The present inventors successfully developed a capsule device to encase a body organ, such as kidney, liver or ovary, or a mass. Using this capsule device allows the treatment or prevention of diseases accompanying abnormal growth of body organs such as polycystic kidney disease. There is no effective treatment for polycystic kidney disease, and the therapeutic method provided by the present invention may be extremely useful.

1. A capsule device comprising a body having an inner cavity for encasing a body organ or mass.

2. The capsule device according to claim 1, wherein the capsule device has a shape approximating to the body organ or mass.

3. The capsule device according to claim 1, wherein the body organ is selected from the group consisting of a kidney, liver, and ovary.

4. The capsule device according to claim 1, wherein the capsule device is used for slowing down or halting growth of the body organ or mass.

5. The capsule device according to claim 1, wherein the capsule device is configured to have an aperture to ensure that a structure connected to the body organ is not interfered with.

6. The capsule device according to claim 1, wherein the body organ is a kidney.

7. The capsule device according to claim 6, wherein the capsule device is used for the treatment or prevention of polycystic kidney disease.

8. (canceled)

9. The capsule device according to claim 6, wherein the capsule device is designed to suppress an increase in total kidney volume.

10. (canceled)

11. (canceled)

12. (canceled)

13. (canceled)

14. The capsule device according to claim 1, wherein bio-compatible material, elastic property, configuration, and/or size of the capsule device are determined based on an individual subject's medical information selected from the group consisting of subject's age, subject's gender, subject's allergic sensitivity profile, target organ's anatomy, and target organ's projected growth rate.

15. The capsule device according to any claim 1, wherein the capsule device is configured to include predetermined surgical opening and closing suture lines within the device by consideration of surgical procedures for the implantation of the capsule device.

16. The capsule device according claim 1, wherein the capsule device is designed to split open at least partially during the placement in order to encase the organ or mass.

17. The capsule device according to claim 16, wherein the capsule device comprises a means to close the split openings of the device.

18. The capsule device according to claim 17, wherein the closing means is selected from the group consisting of interlacing strings, buttons, hooks, fasteners, and hook-and-loop fasteners.

19. The capsule device according to claim 1, wherein the capsule device is made of liquid or flexible injectable material capable of being implanted by minimally-invasive or laparoscopic surgical procedures.

20. (canceled)

21. A method of producing the capsule device according to claim 1, comprising measuring the shape of a body organ or mass of a subject; designing a capsule device adapted for the body organ or mass; and fabricating the capsule device.

22. The method according to claim 21, wherein the measuring is performed using MRI, CT, ultrasound images, fluoroscopic images, or laparoscopic images.

23. The method according to claim 21, wherein the fabricating is performed using 3-D printing or manual fabrication.

24. The method according to claim 21, wherein the designing comprises determining bio-compatible material, elastic property, configuration, and/or size of the capsule device based on an individual subject's medical information selected from the group consisting of: subject's age, subject's gender, subject's allergic sensitivity profile, target organ's anatomy, and target organ's projected growth rate.

25. (canceled)

26. (canceled)

27. (canceled)

28. (canceled)

29. (canceled)

30. A method for the treatment or prevention of abnormal growth of a body organ or mass in a subject in need thereof, comprising implanting the capsule device of claim 1 to encapsulate the body organ or mass of the subject.

31. The method of claim 30, wherein the body organ is selected from the group consisting of a kidney, liver, and ovary.

32. (canceled)

33. (canceled)
